(19) United States
(12) Patent Application Publication
(10) Pub. No.: US 2008/0287746 A1
(43) Pub. Date: Nov. 20, 2008

(54) SYSTEM AND METHOD FOR COMMUNICATING HEALTH CARE ALERTS VIA AN INTERACTIVE PERSONAL HEALTH RECORD

(76) Inventor: Lonny Reisman, Muttontown, NY (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(21) Appl. No.: 11/749,654

(22) Filed: May 16, 2007

(51) Int. Cl. A61B 5/00

(52) U.S. Cl. 600/300

(57) ABSTRACT

An automated system is described for presenting a patient with an interactive personal health record (PHR) capable of delivering individualized alerts based on comparison of evidence-based, best standards of care to information related to the patient's actual medical care. This will assure the patient that the PHR is providing him or her with optimal health care. A health care organization collects and processes a wide spectrum of medical care information, including clinical data relating to a patient and condition-specific medical reference data, in order to generate and deliver customized alerts, including Care Considerations and personalized wellness alerts, directly to the patient via an online interactive personal health record (PHR). In addition to aggregating patient-specific medical record and alert information, the PHR also solicits the patient's input for tracking of alert follow-up actions and allows the health care organization to track alert outcomes.

### FIELD OF THE INVENTION

This invention relates generally to the field of health care management and more specifically to the area of patient health communications.

### BACKGROUND OF THE INVENTION

The health care system includes a variety of participants. In addition to doctors, hospitals, insurance carriers, and patients, there exists a plethora of health care information storage and retrieval systems that are necessary to support a heavy flow of information related to patient care. All participants in the health care system frequently rely on each other for the information necessary to perform their respective roles because individual care is delivered and paid for in numerous locations by individuals and organizations that are typically unrelated. As a result, critical patient data is stored across many different locations using incompatible legacy mainframe and client-server systems that store information in non-standardized formats. To ensure proper patient diagnosis and treatment, health care providers must often request patient information by phone or fax from hospitals, laboratories or other providers. Therefore, disparate systems and information delivery procedures maintained by a number of independent health care system constituents lead to gaps in timely delivery of complete patient records and may compromise the overall quality of clinical care.

Since a typical health care practice is concentrated within a given specialty, an average patient may be using services of a number of different specialists, each potentially having only a partial view of the patient's medical status. To obtain an overview or establish a trend of his or her medical data, a patient (and the patient's primary care physician) is forced to request the medical records separately from each individual health care provider and attempt to reconcile the piecemeal data. Potential gaps in complete medical records further reduce the value of medical advice given to the patient by each health care provider. Existing solutions have generally addressed the problem of centralized storage of health care information, but do little more than store that information and make it available in a presentable form. In particular, these existing solutions do not incorporate analysis of a patient's health care information in order to find medical issues that may require attention. Thus, a need still exists for a personal health record system capable of clinically analyzing the accumulated health care information in light of appropriate medical standards and directly notifying the patient to ensure a prompt follow up on the results of the analysis with a health care provider.

### BRIEF SUMMARY OF THE INVENTION

Embodiments of the invention are used to provide an automated system for presenting a patient with an interactive personal health record powered by clinical decision support technology capable of delivering individualized alerts based on comparison of an expected medical standard of care to information related to the patient's actual medical care. Such embodiments are advantageous over previous, static health record systems that merely store and present health related information. A health care organization or an employer collects and processes a wide spectrum of medical care information, including clinical data relating to a patient and condition-specific medical reference data, in order to generate and deliver customized alerts, including Care Considerations specific to the patient and personalized wellness alerts, directly to the patient via an online interactive personal health record (PHR). In addition to aggregating patient-specific medical record and clinical alert information, the PHR also solicits the patient's input for tracking of alert follow-up actions (such as family history, over-the-counter medications, allergies, herbal supplements, monitoring items such as high blood pressure, cholesterol and diabetic conditions and other elements claims data may not track) and allows the health care organization to track alert outcomes.

A medical insurance carrier typically collects clinical information originating from medical services claims, procedures performed, pharmacy data, lab results, and provides it to the health care organization for storage in a medical database. The medical database comprises one or more medical data files located on a computer readable medium, such as a hard disk drive, a CD-ROM, a tape drive, or the like.

In addition to collecting the claims, procedures, pharmacy and lab results derived clinical data, the health care organization communicates with a plurality of sources of medical care information to collect health reference information, as well as medical news and other related information corresponding to a wealth of known and newly-discovered medical conditions. An on-staff team of medical professionals within the health care organization consults various sources, including collected health reference and medical news information, to establish and continuously revise a set of rules that reflect the best medical standards of care for a plurality of conditions. The rules are stored in the medical database.

To supplement the clinical data received from the insurance carrier, the PHR allows patient entry of additional pertinent medical information that is likely to be within the realm of patient's knowledge, including family history, use of non-prescription drugs, known allergies, unreported and untreated conditions, as well as results of self-administered medical tests. Preferably, the PHR facilitates patient's task of creating a complete health record by automatically populating the data fields corresponding to the information derived from the claim, pharmacy and/or lab result-based clinical data. Preferably, the PHR gathers at least some of the user-entered data via a health risk assessment tool (HRA) that allows user entry of family history, known chronic conditions and other medical data, to provide overall patient health assessment or to flag individuals at risk for one or more predetermined medical conditions. Preferably, the HRA tool presents a patient with questions that are relevant to his or her medical history and currently presented conditions. The risk assessment logic branches dynamically to relevant and/or critical questions, thereby saving the patient time and providing targeted results. The data entered by the patient into the HRA also populates the corresponding data fields within other areas of PHR and generates additional Care Considerations to assist the patient in maintaining optimum health.

The health care organization aggregates the medical care information, the user-entered data and insurance clinical data into the medical database for subsequent processing via an analytical system such as the CareEngine® System operated by ActiveHealth Management, Inc., of New York, N.Y. The CareEngine® System is a multidimensional analytical software tool comprising computer readable instructions for applying a set of rules that reflect the best evidence-based medical standards of care for a plurality of conditions and compares this to the actual care that is being delivered by caregivers to the patient. The CareEngine® System identifies one or more instances where the patient's actual care, as evidenced by claims data (including medical procedures, tests, pharmacy data and lab results) and typically user-entered data, is inconsistent with the best evidence-based standards of care. Additionally, the CareEngine® System applies specific rules to determine when the patient should be notified of newly available health reference information to provide the best in care. In addition to analyzing the medical procedures, tests, pharmacy claims and lab results, the CareEngine® System analyzes known allergies, chronic conditions, untreated conditions and other patient-reported clinical data to process and issue condition-specific alerts (called Care Considerations) directly to the patient via a set of Web pages comprising the PHR tool. With the consent of the patient, the physician can have access to the Web pages, also.

To ensure prompt patient response, the health care organization preferably sends concurrent email notifications to the patient regarding availability of customized alerts at the PHR. When the CareEngine® System identifies an instance of actual care inconsistent with the established, evidence-based best standards of care, the patient is presented with a Care Consideration alert via the online PHR. In embodiments, the Care Considerations include notifications to contact the health care provider in order to start or stop a specific medication and/or to undergo a specific examination or test procedure associated with one or more conditions and co-morbidities specific to the patient. The Care Consideration includes notifying the patient regarding known drug interactions and newly suggested medications based on the evidence-based best practices of care. Similarly, the CareEngine® System notifies the patient regarding relevant health reference information by issuing personalized wellness alerts, via the PHR, based on analyzing the newly acquired health reference information with respect to insurance-based clinical data and user-entered data to ensure overall consistency of care. In one embodiment, the patient is able to use the PHR to search for specific health reference information regarding a specified condition, test or medical procedure by querying the medical database via a user interface. Preferably, the PHR allows the patient to create printable reports containing the patient's health information, including health summary and health risk assessment reports, for sharing with a health care provider.

Additionally, by functioning as a central repository of a patient's medical information, the PHR empowers patients to more easily manage their own health care decisions, which is advantageous as patients increasingly move toward consumer-directed health plans.

### BRIEF DESCRIPTION OF THE DRAWINGS

While the appended claims set forth the features of the present invention with particularity, the invention and its advantages are best understood from the following detailed description taken in conjunction with the accompanying drawings, of which:

FIG. 1 is a schematic illustrating an overview of a system for presenting a patient with a personal health record capable of delivering medical alerts, in accordance with an embodiment of the invention;

FIG. 2 is a flow diagram illustrating a method for providing a customized alert to a patient, in accordance with an embodiment of the invention;

FIG. 3 is a diagram of a user interface presented by a main page of the Web-based Personal Health Record (PHR) tool of FIG. 1, in accordance with an embodiment of the invention;

FIG. 4 is a diagram of a user interface presented by an alerts detail page of the PHR tool of FIG. 1, in accordance with an embodiment of the invention;

FIGS. 4A-4B are user interface diagrams of an alternative embodiment of the alerts detail page of FIG. 4, including an embodiment of the reviewed alerts report;

FIG. 5 is a diagram of a user interface of a Health Risk Assessment (HRA) questionnaire of the PHR tool of FIG. 1, in accordance with an embodiment of the invention;

FIG. 6 is a diagram of a conditions and symptoms interface associated with the HRA of FIG. 5, in accordance with an embodiment of the invention;

FIG. 7 is a diagram of a family history interface associated with the HRA of FIG. 5, in accordance with an embodiment of the invention;

FIGS. 8-12 are diagrams of additional user interfaces of the PHR tool of FIG. 1 permitting patient entry of information relating to medications, allergies, immunizations, tests, and hospital visits, in accordance with an embodiment of the invention;

FIG. 13 is a diagram of a health summary interface presenting the patient with a summary of health care information available via interfaces of FIGS. 5-12, in accordance with an embodiment of the invention;

FIG. 14 is a diagram of an emergency information card generated based on at least some of the information available via the Web-based PHR tool of FIG. 1, in accordance with an embodiment of the invention;

FIG. 15 is a diagram of a health care team interface page of the Web-based PHR tool of FIG. 1, in accordance with an embodiment of the invention;

FIG. 16 is a diagram of a health care tracking tool available to the patient via the Web-based PHR of FIG. 1, in accordance with an embodiment of the invention; and

FIG. 17 is a diagram of a graphical output of a Care Consideration Alert Status report indicating the alert completion and outcome status for the overall patient population, in accordance with an embodiment of the invention.

### DETAILED DESCRIPTION OF THE INVENTION

The following examples further illustrate the invention but, of course, should not be construed as in any way limiting its scope.

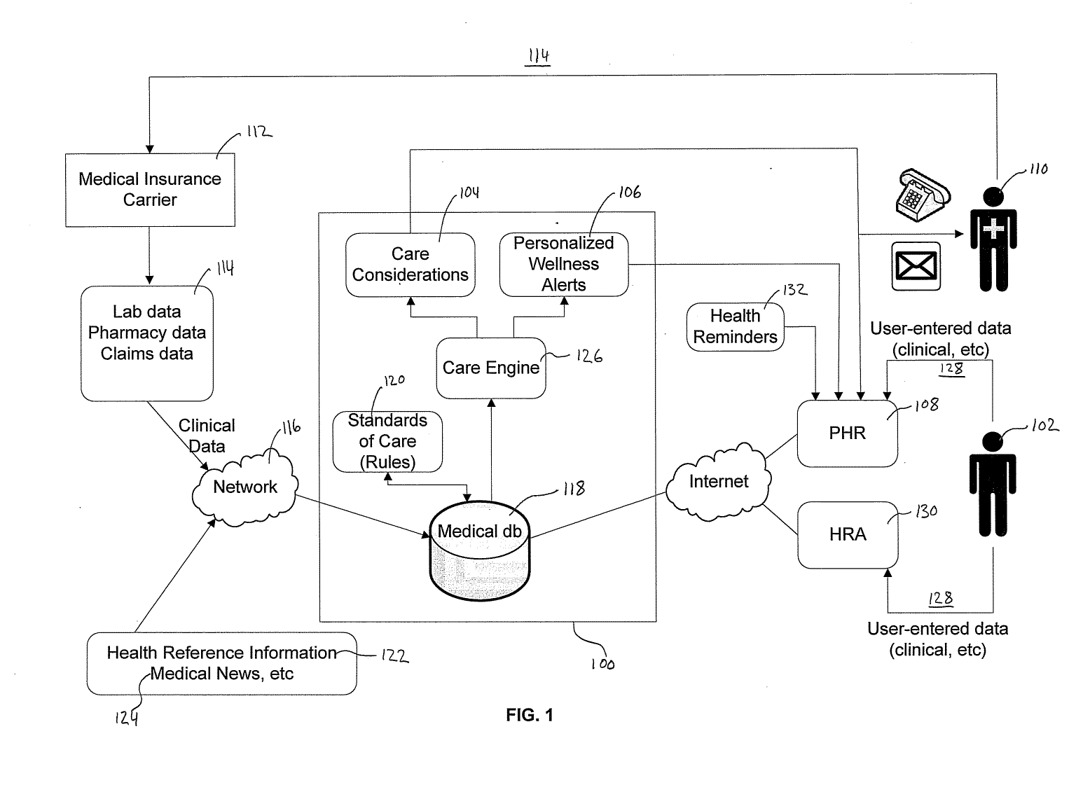

Turning to FIG. 1, an implementation of a system contemplated by an embodiment of the invention is shown with reference to an automated system for presenting a patient with an interactive personal health record powered by clinical decision support technology capable of delivering individualized alerts (including those called Care Considerations) based on comparison of the best evidence-based standards of care to a patient's actual medical care. The health care organization 100 collects and processes a wide spectrum of medical care information, including clinical data relating to a patient 102 and condition-specific medical reference data, in order to generate and deliver customized alerts, including Care Considerations 104 and personalized wellness alerts 106, directly to the patient 102 via an online interactive personal health record (PHR) represented by PHR 108. In addition to aggregating patient-specific medical records and alert information, as well as other functionality to be discussed herein, the PHR 108 also solicits the patient's input for tracking of alert follow-up actions and allows the health care organization 100 to track alert outcomes.

When the patient 102 utilizes the services of one or more health care providers 110, a medical insurance carrier 112 typically collects the associated clinical data 114 in order to administer the health insurance coverage for the patient 102. Clinical data 114 originates from medical services claims, pharmacy data, as well as from lab results generated pursuant to the patient-health care provider interactions and includes information related to the patient's diagnosis and treatment, including medical procedures, drug prescription information, in-patient information and health care provider notes. The medical insurance carrier 112, in turn, provides the clinical data 114 to the health care organization 100, via the network 116, for storage in a medical database 118. The medical database 118 is administered by one or more backend computers (not shown) associated with the health care provider 100 and comprises one or more medical data files located on a computer readable medium, such as a hard disk drive, a CD-ROM, a tape drive or the like. The medical database 118 preferably includes a commercially available database software application capable of interfacing with other applications, running on the same or different backend computer, via a standard query language (SQL). In an embodiment, the network 116 is a dedicated medical records network. Alternatively or in addition, the network 116 includes an Internet connection which comprises all or part of the network.

In addition to collecting the medical claims, pharmacy and lab result data derived from the clinical data 114, the health care organization 100 communicates with a plurality of sources of medical care information via the network 116 to collect the health reference information 122, as well as medical news and other related information 124 corresponding to a plurality of known and newly-discovered medical conditions. In an embodiment, an on-staff team of medical professionals within the health care organization 100 consults various sources, including collected health reference information 122 and medical news information 124, to establish and continuously or periodically revise a set of rules 120 that reflect medical standards of care for a plurality of conditions. The rules 120 are stored in the medical database 118.

To supplement the clinical data 114 received from the insurance carrier 112, the PHR 108 allows patient entry of additional pertinent medical information that is likely to be within the realm of patient's knowledge. Exemplary user-entered data 128 includes additional clinical data, such as patient's family history, use of non-prescription drugs, known allergies, unreported and/or untreated conditions (e.g., chronic low back pain, migraines, etc.), as well as results of self-administered medical tests (e.g., periodic blood pressure and/or blood sugar readings). Preferably, PHR 108 facilitates the patient's task of creating a complete health record by automatically populating the data fields corresponding to the information derived from the medical claims, pharmacy data and lab result-based clinical data 114. In one embodiment, user-entered data 128 also includes non-clinical data, such as upcoming doctor's appointments. Preferably, the PHR 108 gathers at least some of the user-entered data 128 via a health risk assessment tool (HRA) 130 that allows user entry of family history, known chronic conditions (e.g., chronic back pain, migraines) and other medical data, to flag individuals at risk for one or more predetermined medical conditions (e.g., predetermined chronic diseases, heart disease, diabetes, risk of stroke) pursuant to the processing by the CareEngine® System 126. Preferably, the HRA 130 tool presents the patient 102 with questions that are relevant to his or her medical history and currently presented conditions. The risk assessment logic branches dynamically to relevant and/or critical questions, thereby saving the patient time and providing targeted results. The data entered by the patient 102 into the HRA 130 also populates the corresponding data fields within other areas of PHR 108. The health care organization 100 aggregates the medical care information 122-124, the user-entered data 128 and the clinical data 114 into the medical database 118 for subsequent processing via the CareEngine® System 126.

The CareEngine® System 126 is a multidimensional analytical software tool comprising computer readable instructions for applying a set of rules 120 to the contents of the medical database 118 in order to identify an instance where the patient's 102 actual care, as evidenced by the clinical data 114 and the user-entered data 128, is inconsistent with the best evidence-based standards of care. Additionally, the CareEngine® System 126 applies condition-specific rules 120 to determine when the patient 102 should be notified of newly available health reference information, which enhances the patient's individual involvement in health care decisions. After collecting the relevant data 114 and 128 associated with the patient 102, the CareEngine® System 126 applies the rules 120 specific to the patient's medical data file, including checking for known drug interactions, to compare the patient's actual care with the best, evidence-based medical standard of care. In addition to analyzing the claims and lab result-derived clinical data 114, the analysis includes taking into account known allergies, chronic conditions, untreated conditions and other patient-reported clinical data to process and issue condition-specific alerts 104 and 106 directly to the patient 102 via a set of Web pages comprising the PHR tool 108. The CareEngine® System process 126 is executed by a backend computer in communication with the medical database 118. In one embodiment, the computer readable instructions comprising the CareEngine® System 126 and the medical database 118 reside on a computer readable medium of a single computer controlled by the health care organization 100 Alternatively, the CareEngine® System 126 and the medical database 118 are interfacing via separate computers controlled by the health care organization 100, either directly or through a network. Additional details related to the processing techniques employed by the CareEngine® System 126 are described in U.S. Pat. No. 6,802,810 to Ciarniello, Reisman and Blanksteen, which is incorporated herein by reference in its entirety.

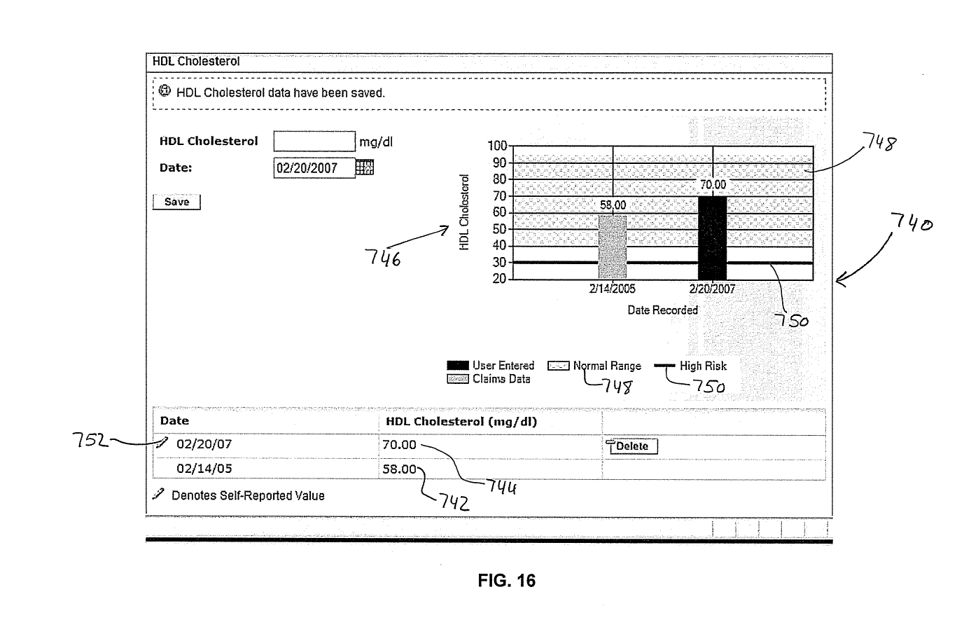

To ensure prompt patient response, the health care organization 100 preferably sends concurrent email notifications to the patient 102 regarding availability of customized alerts 104 and 106 at the PHR 108. As described herein, the terms “alerts” and “customized alerts” refer to condition-specific patient notifications, such as Care Considerations 104 and personalized wellness alerts 106, which have been delivered directly to the patient 102 via the PHR 108 after being generated by the CareEngine® System 126 pursuant to the processing of one or more of the clinical data 114, user-entered data 128, health reference information 122 and medical news 124, and flagged as evidence-based, best standards of care defined by the CareEngine® rules 120. When the CareEngine® System 126 identifies an instance of actual care which is inconsistent with evidence-based, best standards of care 120, the patient 102 is presented with a Care Consideration 104 via the online PHR 108. Preferably, the Care Considerations 104 are prominently displayed within a user interface of the PHR 108. In embodiments, the Care Considerations 104 include notifications to contact the health care provider 110 in order to start or stop a specific medication and to undergo a specific test procedure associated with one or more conditions and co-morbidities specific to the patient 102. The Care Considerations 104 include notifying the patient regarding known drug interactions and newly suggested medications derived from the current medical standard of care information 120. The Care Considerations 104 are also prompted by analysis of patient's medication regimen in light of new conditions and lab results. Similarly, the CareEngine® System 126 notifies the patient 102 regarding the relevant health reference information 122 by issuing personalized wellness alerts 106, via the PHR 108, based on analyzing the newly acquired health reference information 122 with respect to the clinical, pharmacy and lab data 114 and user-entered data 128 to ensure overall consistency of care. In one embodiment, the patient 102 is able to use the PHR 108 to search for specific health reference information regarding a specified condition, test or medical procedure by querying the medical database 118 via a user interface. In another embodiment, the patient 102 subscribes to medical news information 124 for delivery via the PHR 108 and/or personal email. In yet another embodiment, the patient 102 receives general health reminders 132 based on non-clinical components of the user-entered data 128 that are not processed by the CareEngine® System 126, such as notifications regarding upcoming doctor appointments. In embodiments, the general health reminders 132 include prompting the patient 102 to update the HRA 130, watch a video tour of the PHR website, or update the health tracking information (discussed below in connection with FIG. 16). Preferably, the PHR 108 allows the patient 102 to create printable reports containing the patient's health information, including health summaries and health risk assessment reports, for sharing with the health care provider 110.

To ensure further follow-up, the health care organization 100 optionally notifies the health care provider 110 regarding the outstanding Care Consideration 104, as disclosed in the incorporated U.S. Pat. No. 6,802,810. For example, if a Care Consideration 104 includes a severe drug interaction, the health care organization 100 prompts the health care provider 110, via mail, email, phone or other communications, to initiate immediate follow-up.

While the entity relationships described above are representative, those skilled in the art will realize that alternate arrangements are possible. In one embodiment, for example, the health care organization 100 and the medical insurance carrier 112 is the same entity. Alternatively, the health care organization 100 is an independent service provider engaged in collecting, aggregating and processing medical care data from a plurality of sources to provide a personal health record (PHR) service for one or more medical insurance carriers 112. In yet another embodiment, the health care organization 100 provides PHR services to one or more employers by collecting data from one or more medical insurance carriers 112.

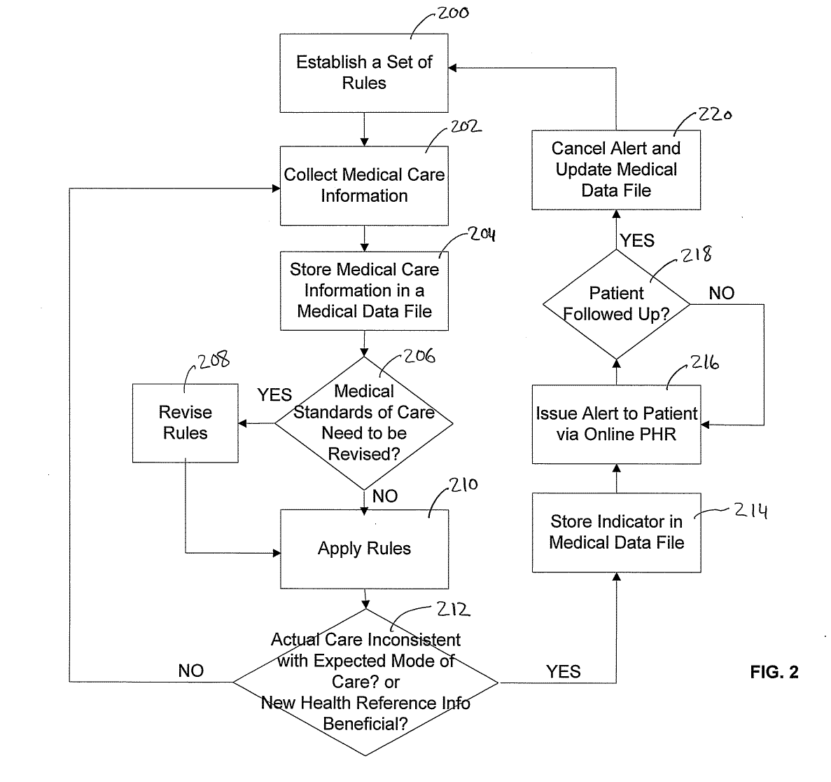

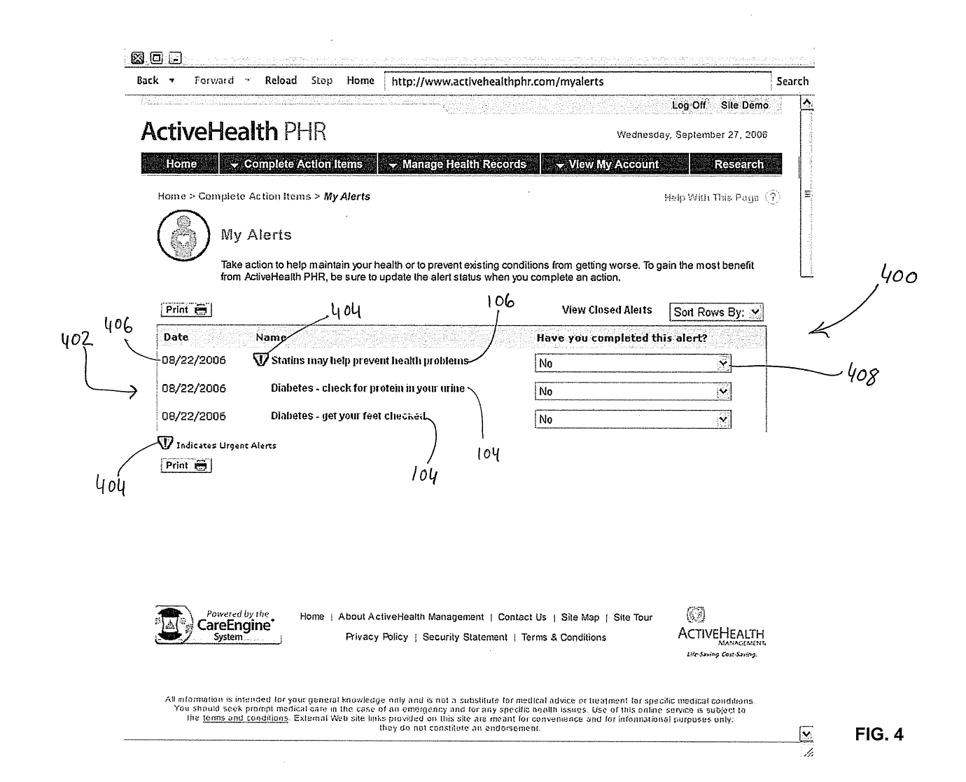

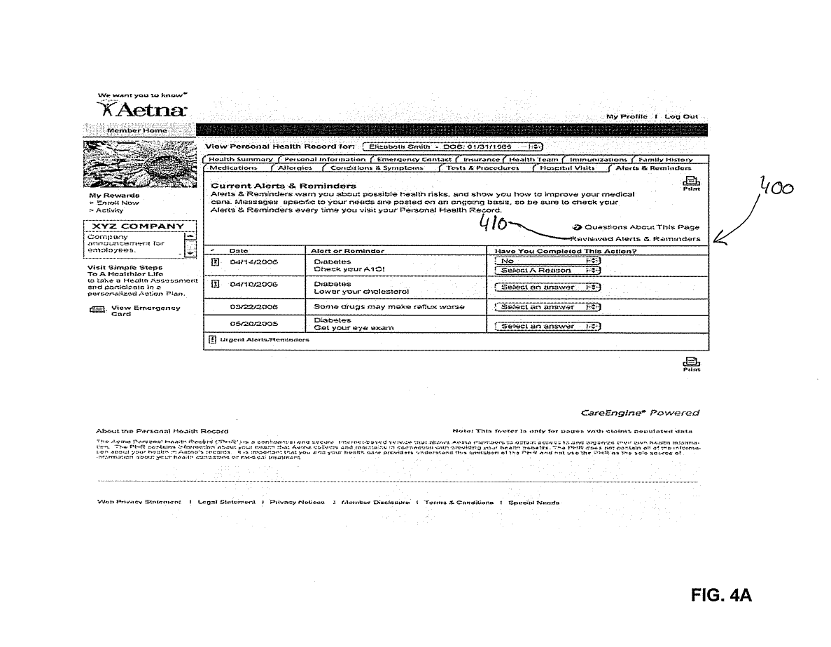

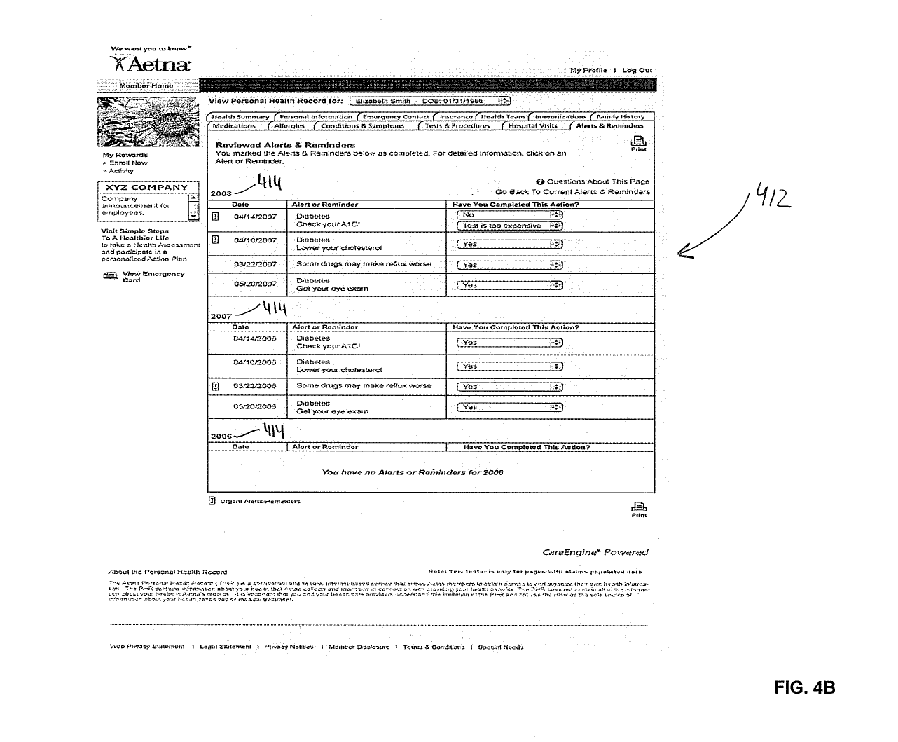

Turning to FIG. 2, a method for providing customized alerts to an individual patient via a personal health record is described. In step 200-204, the health care organization 100 establishes a set of rules 120 for a plurality of conditions by continuously collecting medical care information and aggregating it in the medical database 118. See FIG. 1. When updates to the medical standards of care become necessary, such as due to updated health reference information 122 becoming available at the medical database 118, the health care organization 100 revises the rules 120 associated with evidence-based, best standards of care. See FIG. 1. In steps 206 and 208, evidence-based medical standards of care will have to be revised. In steps 210 and 212, the CareEngine® System 126 applies the latest evidence-based, best standard rules 120 to the patient's actual care, as evidenced from the claims, pharmacy, lab and user-entered clinical data, to identify at least one instance where the patient's evidence-based, best standards of care is inconsistent with the expected care embodied by the rules 120. See FIG. 1. Alternatively or in addition, step 212 includes identifying whether the patient 102 should be notified about newly available health reference information 122, such as when the health reference information is beneficial to the patient's evidence-based, best standards of care reflected in claims, pharmacy, lab and user-entered clinical data. See FIG. 1. If the CareEngine® System process 126 does not detect a discrepancy between the actual care given by the caregiver and the evidence-based, best standards of care, or when the newly received health reference is not beneficial (e.g., cumulative in light of existing information). See FIG. 1. In this particular case, the method returns to step 202. Otherwise, in steps 214-216, the CareEngine® System process 126 stores an alert indicator in the patient's 102 medical data file within the medical database 118, including the associated alert detail, and presents the patient with one or more alerts 104 and 106 via the appropriate interface of the online PHR 108. See FIG. 1. Optionally, the CareEngine® System 126 notifies the patient 102, via email or otherwise, to log into the PHR 108 in order to view one or more issued alerts (called Care Considerations) in 104 and 106. See FIG. 1. As discussed in further detail in connection with FIG. 4 below, the PHR 108 provides the patient 102 with an opportunity to update the system with status or outcome of the alert follow-up. See FIG. 1. To that end, if the patient 102 indicates that the alert has been addressed, the PHR 108 will cancel the corresponding alert indicator and update the medical database 118 with the follow-up status or outcome. See FIG. 1. In steps 218 and 220, the patient is issued a Care Consideration. Alternatively or in addition, the system updates an alert indicator based on becoming aware of alert follow-up via changes in claim data. Otherwise, the PHR 108 continues to prompt the patient 102 to follow-up on the alert. See FIG. 1.

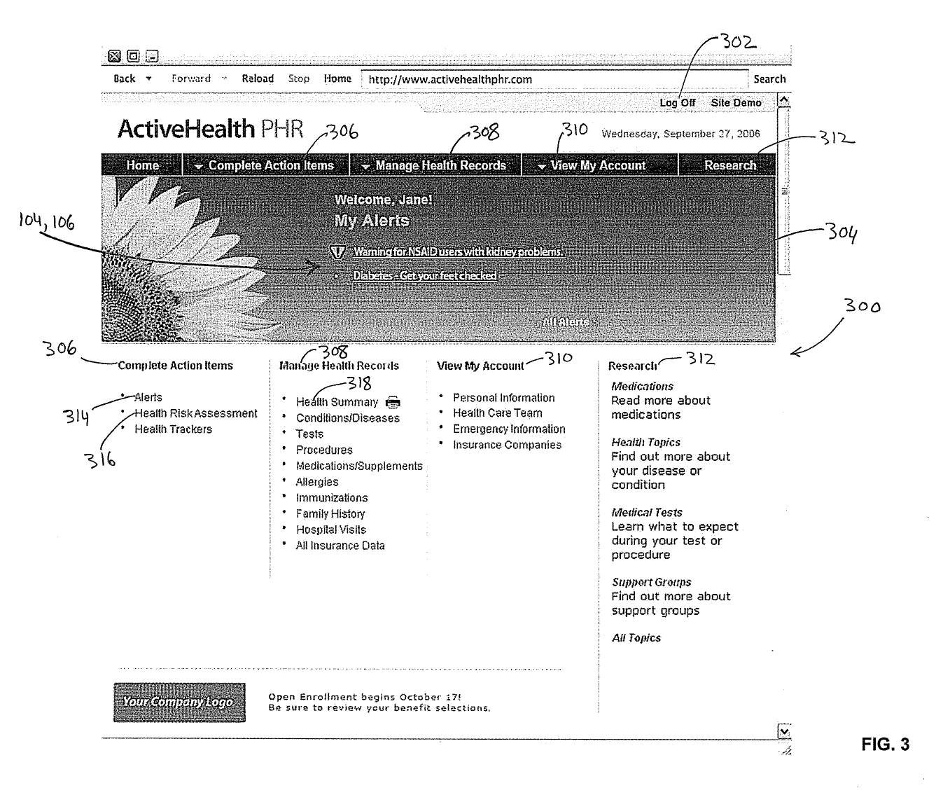

FIGS. 3-17 below provide additional detail regarding various embodiments of the PHR 108 and its associated functionality. Turning to FIG. 3, an embodiment of the main page 300 of the Web-based PHR 108 is shown. In one embodiment, when the patient 102 obtains access to the PHR 108 via a secure login/logoff area 302, the PHR 108 presents the patient with an alert display area 304 having one or more selectable alerts 104 and 106 which are awaiting the patient's follow-up. The main page 300 further includes a plurality of links generally related to alert follow-up and health risk assessment (HRA) 306, health record management 308, account administration 310 and online health library access 312. While the PHR 108 pre-populates some patient information using the clinical data received from the medical insurance carrier 112, user-entered data comprises an important part of the overall record. Therefore, embodiments of the invention include providing incentives to the patient 102 in order to elicit a complete response to the user-entered data fields, such as those in a Health Risk Assessment (HRA) tool 130 and, optionally, to ensure alert follow-up. In one embodiment, the incentives include a points program administered by the patient's employer or by the health care organization 100.

Upon selecting the alerts link 314 or any of the pending alerts 104 and 106 displayed in the alerts display area 304, the patient 102 is directed to the alerts detail page 400, as illustrated in FIG. 4. The alerts detail page 400 presents the patient with an alerts list 402, which includes alerts pending the patient's follow-up and is preferably pre-sorted by urgency level 404 and notification date 406. In the illustrated embodiment of FIG. 4, the alerts list 402 includes a wellness alert 404 notifying the patient of relevant health reference information, which in this case indicates that statins may help prevent health problems as well as with a pair of Care Considerations 104 suggesting specific tests related to patient's diabetes. The list 402 further includes an alert completion status dropdown list 408 to provide the health care organization 100 with follow-up status as to the issued alerts 104 and 106. The alert completion status dropdown list 408 allows the patient 102 to indicate whether a specific alert has been completed and, if so, to select additional detail related to the completion outcome. In this embodiment, the dropdown list 408 includes choices indicating that the patient has contacted the health care provider 110 to either start or stop the flagged medication, complete the flagged test and/or discuss the flagged health reference information. Additionally, the list 408 allows the patient to provide reasons for not completing a pending alert, such as by indicating that the patient is still planning to discuss the alert with the health care provider 110, that the patient is allergic or otherwise intolerant to the suggested medication or test procedure, that the patient cannot afford the suggested treatment or that the alert is otherwise not applicable. Another embodiment of the alerts detail page 400 is illustrated in FIG. 4A wherein the alerts interface 400 includes a reviewed alerts link 410 to allow the patient 102 to view and update previously reviewed alerts. The reviewed alerts link 410 is associated with a reviewed alerts report 412 (FIG. 4B) presenting the patient 102 with a list of previously reviewed alerts sorted by year 414.

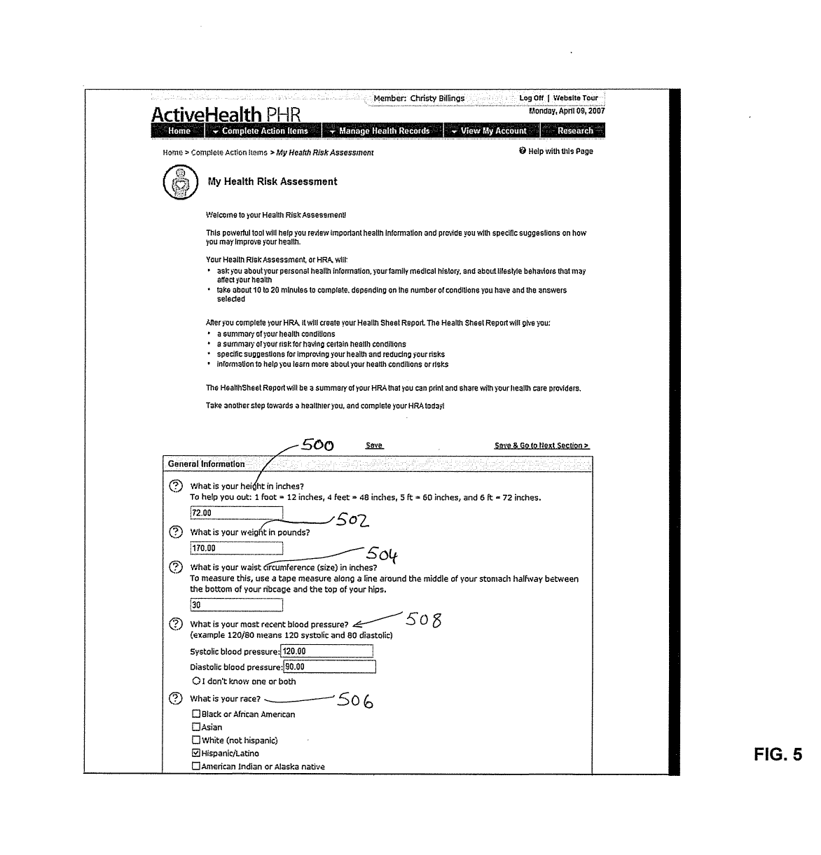

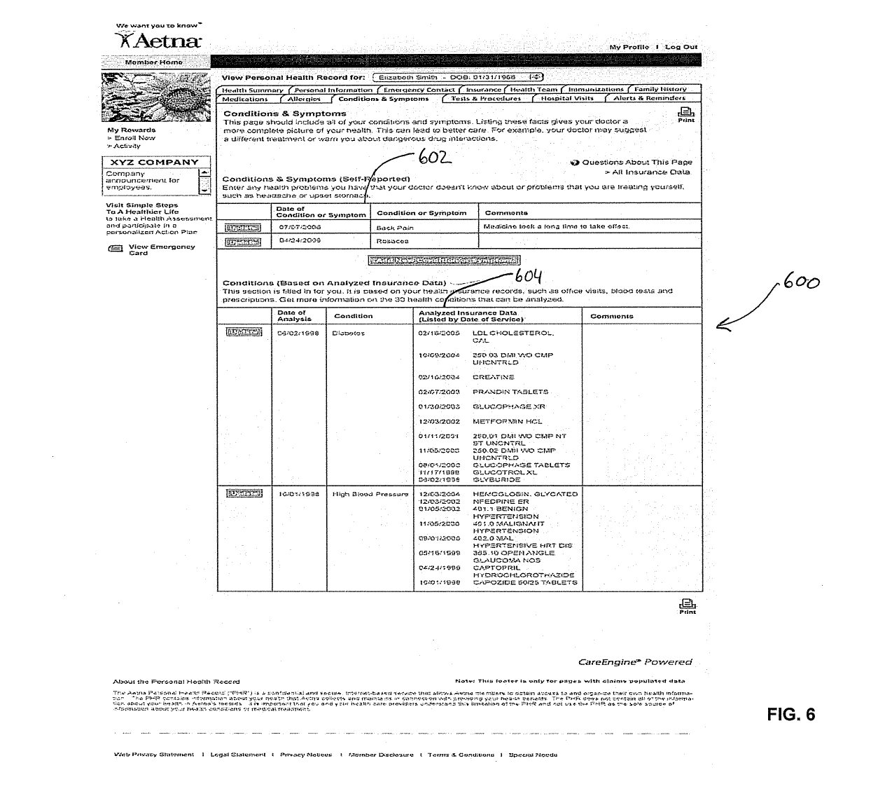

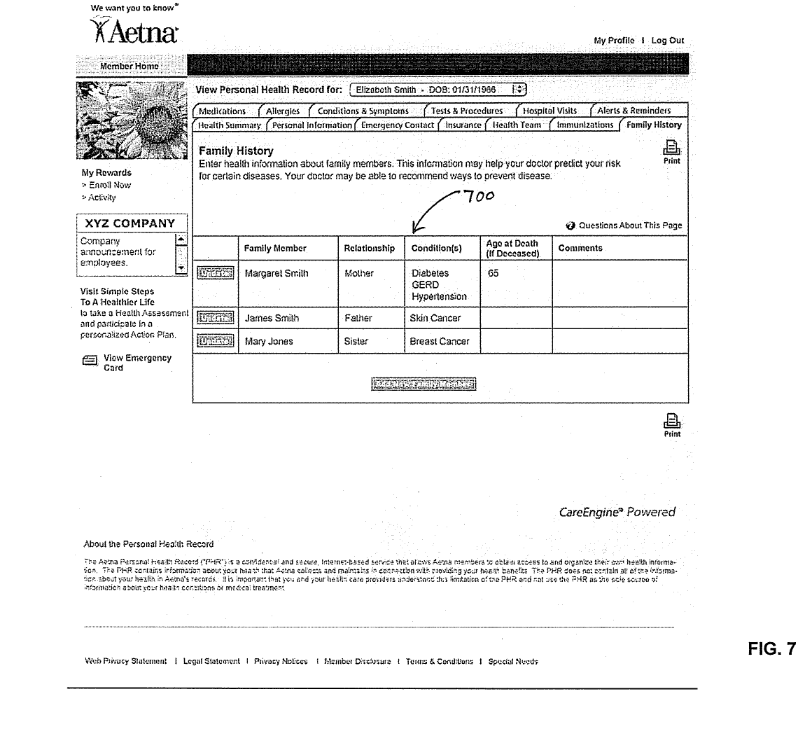

The PHR 108 main page 300 (FIG. 3) also includes a link 316 to the HRA tool 130, which allows the health care organization 100 to gather additional data 128 from the patient 102 to perform CareEngine® System analysis for identifying individuals at risk for one or more predetermined medical conditions. As illustrated in FIGS. 5-7, the HRA 130 combines clinical data derived from health insurance carrier 112 with patient-entered personal health information, family medical history, unreported medical conditions, lifestyle behaviors, and other information to provide the patient 102 with specific health improvement suggestions via care consideration alerts 104 and personalized wellness alerts 106. As seen in FIG. 5, the HRA interface 130 initially prompts the patient 102 to enter general information, such as height 500, weight 502, waist circumference 504, race 506, and recent blood pressure readings 508 prior to presenting the patient 102 with a conditions and symptoms interface 600 (FIG. 6). The conditions and symptoms interface 600, in turn, allows the patient 102 to enter health problems 602 that the health care provider 110 is not aware of and/or health problems which the patient 102 is self-treating, such as upset stomach, back pain, or a headache. Preferably, the HRA 130 also allows the patient to view and update pre-populated conditions 604 based on insurance carrier clinical data 114 previously validated and analyzed by the care engine 126. In one embodiment, the patient 102 is able to opt out from displaying at least some conditions within the conditions and symptoms interface 600, such as to provide a health care provider 110 with a customized printout of patient's conditions. As shown in FIG. 7, patient-entered family history information 700 helps predict the risk associated with certain hereditary diseases. Information entered into the HRA 130 cross-populates other areas of the PHR 108 and vice-versa.

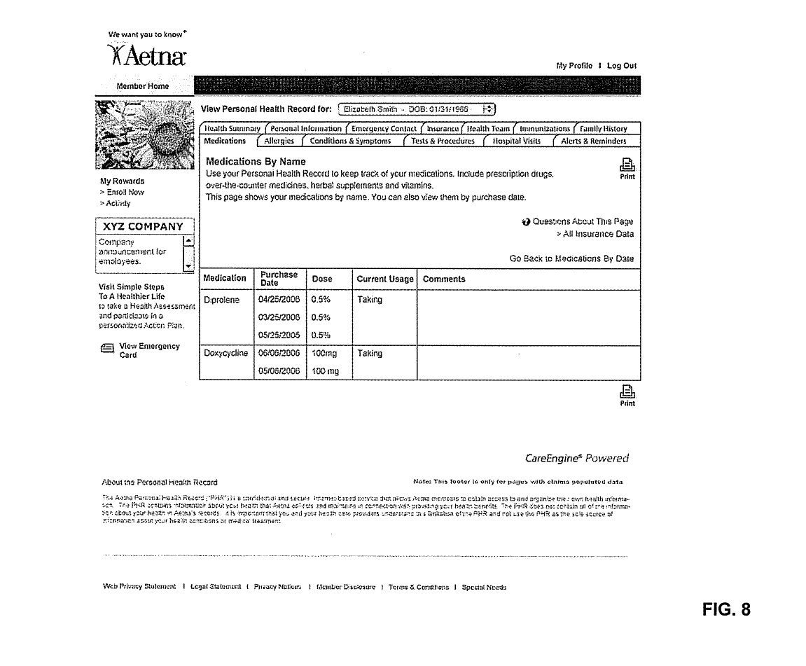

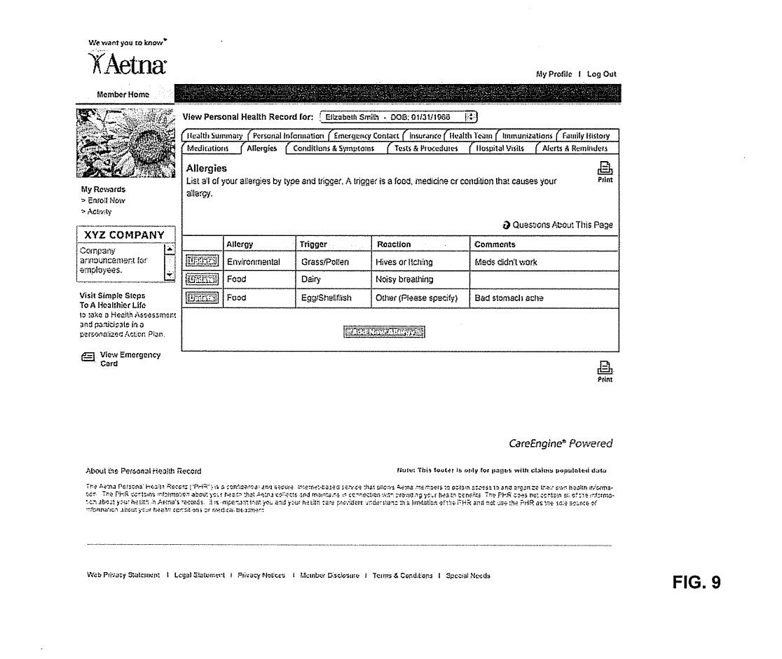

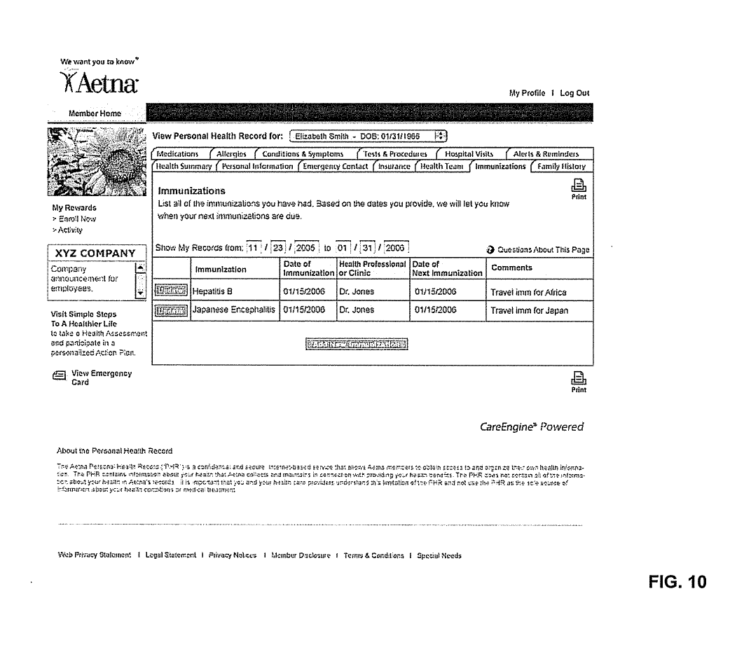

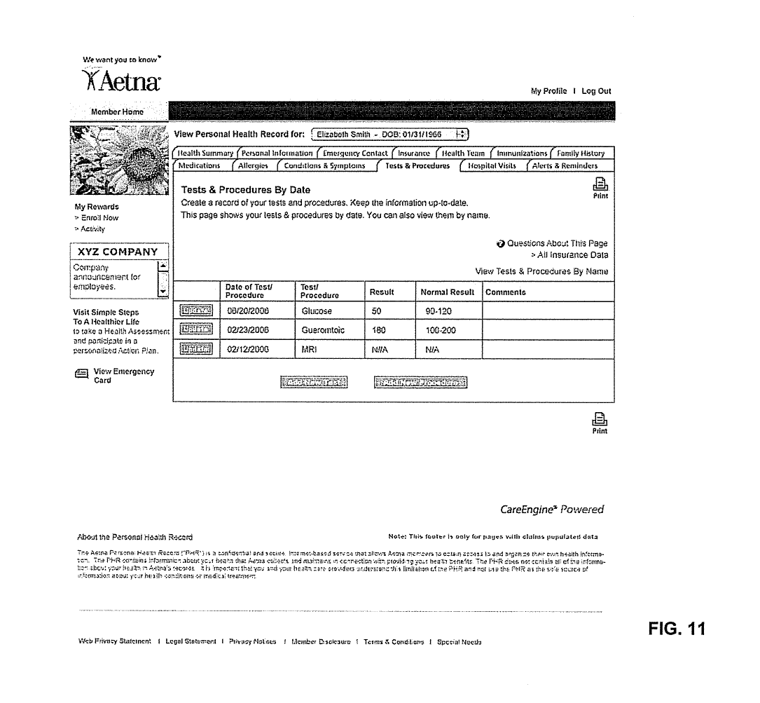

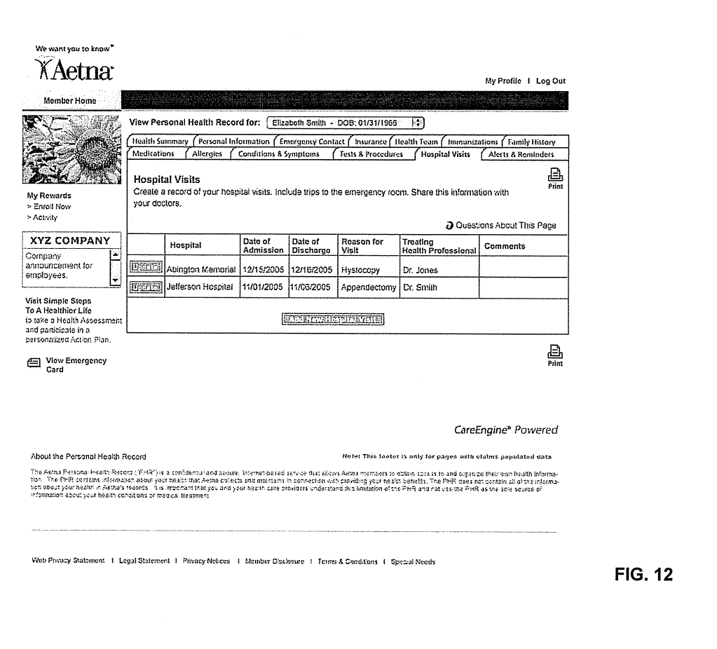

As illustrated in FIGS. 8-12, other areas of PHR 108 permit the patient 102 to enter and view prescription and non-prescription medication and supplements (FIG. 8), list allergies and associated allergy triggers (FIG. 9), update an immunization list (FIG. 10), and create a record of tests, procedures, and hospital visits (FIGS. 11, 12).

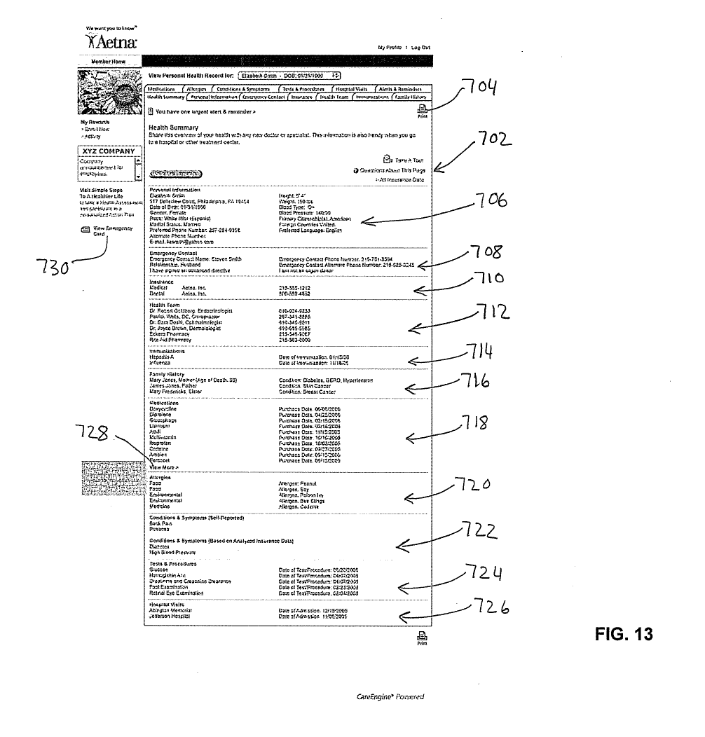

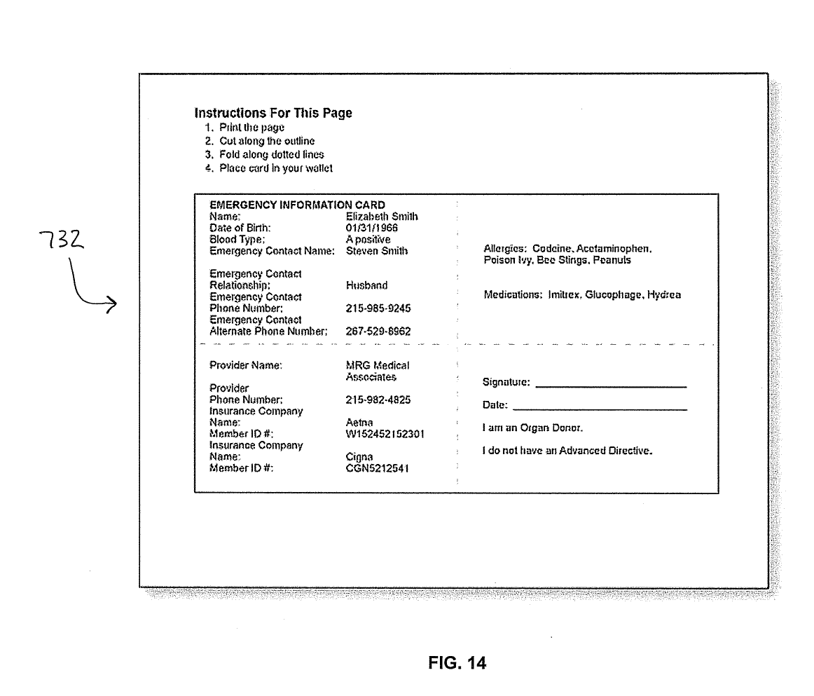

To view a summary of some or all of the information available via FIGS. 5-12, the PHR 108 includes a link 318 (FIG. 3) to a health summary page 702. As shown in FIG. 13, the health summary interface 702 includes a print button 704 that allows the patient 102 to share an overview of his or her health with a health care provider 110 during visits to the doctor's office or hospital. The health summary 702 includes both claim-derived and user-entered data. Specifically, the health summary 702 includes the following information: patient's personal information 704, emergency contacts 708, insurance provider contact information 710, health team 712 (such as treating physicians and preferred pharmacies), immunizations 714, family history 716, prescription and over-the-counter medications 718, allergies 720, conditions and symptoms 722 (including conditions based on insurance claims data analyzed by the care engine 126, as well as self-reported data), as well as test procedures and hospital visit information 724, 726. The “view more” link 728 allows the patient 102 to drill down and include more data, such as when the medications list 718 includes additional medication history. Conversely, the PHR 108 also allows the patient 102 to opt out from displaying at least some of the information in the health summary 702, so as to tailor the type of information displayed in this report for a specific health care provider 110, or to edit out certain sensitive information. In one embodiment, the PHR 108 allows the patient 102 to opt out from displaying some or all patient-entered information in the health summary 702, while always displaying the claim-derived data. Alternatively or in addition, the patient 102 is able to print some or all sections 706-726 of the health summary 702 for sharing with the health care provider 110. As all other information comprising the PHR 108, information that the patient 102 opts not to display in the health care summary 702 remains stored in the medical database 118 (FIG. 1) and available to the care engine 126 for deriving care considerations 104 and personalized wellness alerts 106. Furthermore, such information remains available for patient's viewing via other areas of the PHR 108, as described above in connection with FIGS. 5-12. As a further advantage, a more condensed summary of the information available via PHR 108 is available to the patient 102 via the link 730 in form of an emergency information card 732 (FIG. 14).

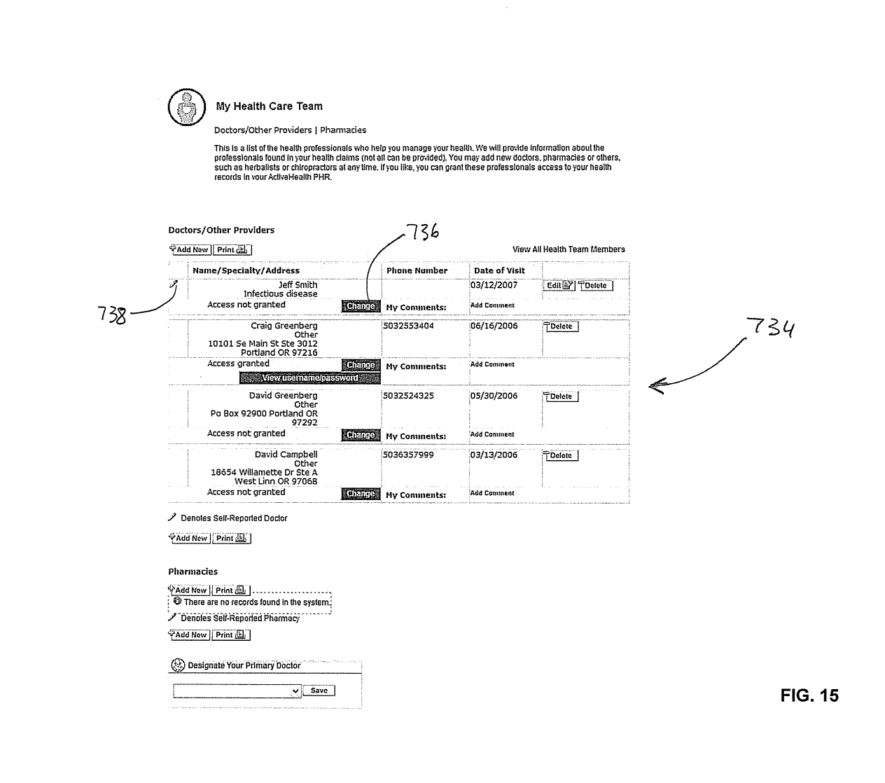

Preferably, the patient 102 supplements the health team list 712 via a health care team page 734, as shown in FIG. 15. The health care team page 734 allows the patient 102 to add new doctors, pharmacies, chiropractors, other health care providers, and designate a primary doctor at any time without waiting for the claim-populated information. Preferably, the patient 102 controls a health care provider's read and/or write access to the PHR 108 by assigning username and password to the provider of choice via the access button 736. The self-reported indicator 738 designates a self-reported health care provider for patient's reference. In embodiments, the patient 102 allows one or more health care providers access to some or all of the information available via the PHR 108. Other embodiments include allowing family member or caregiver access to the PHR 108, as well as providing the patient 102 with access to personal health record information of a dependent. In yet another embodiment, the PHR 108 provides the patient 102 with a data import/export utility capable of porting the information comprising the PHR 108 between health care providers. Additional embodiments include allowing the patient 102 to delete the display of at least some health care providers from the list 712.

Turning to FIG. 16, the PHR 108 further includes a health tracking tool 740 to allow the patient 102 to trend one or more health indicators. In the illustrated embodiment, the health tracking tool 740 combines the claims data 742 with patient-reported data 744 (e.g., from the HRA 130 of FIG. 5) to provide the patient 102 with a graphical representation 746 of an HDL cholesterol trend. Additional embodiments of the health tracking tool 740 include tracking other health indicators capable of periodic evaluation, such as blood pressure, for example. Preferably, the graphical representation area 746 includes normal range and high risk indicators 748, 750 to provide the patient 102 with a health risk assessment trend. Self-reported values are represented via a self-reported indicator 752.

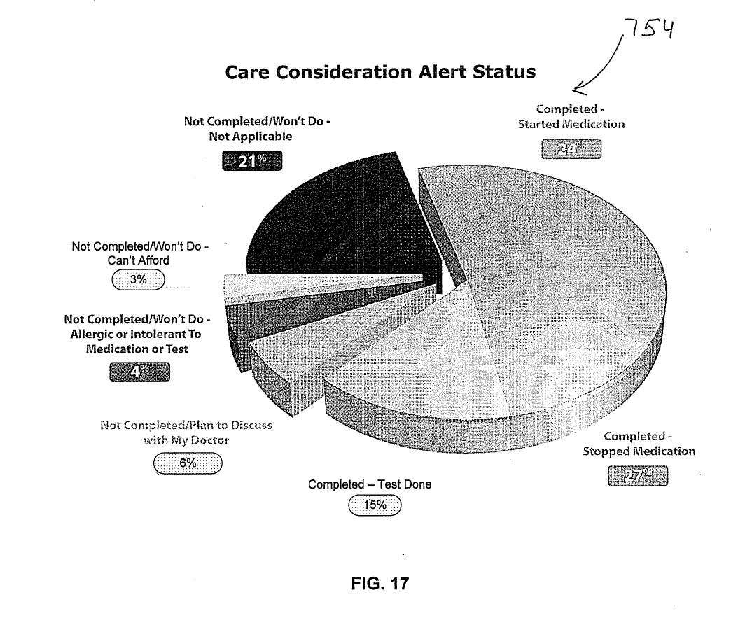

As shown in FIG. 17, the health care organization 100 tracks the alert outcome for the overall patient population by querying the patient-entered alert status stored in the medical database 118 (See FIG. 1). In the illustrated embodiment, the Care Consideration alert status report 754 indicates the alert completion status for the overall patient population as selected by each individual patient 102 via the alert completion status dropdown list 408 (See FIG. 4) of the Web-based PHR 108. Other embodiments include providing PHR utilization reports to employers for gauging employee participation.

Additional embodiments of the PHR 108 include using the PHR interface to display employer messages, as well as providing secure messaging between the patient 102 and a health care provider 110 via the PHR.

All references, including publications, patent applications and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of providing a customized alert to an individual patient via a personal health record comprising:
establishing a set of rules from available medical standards, at least one such rule defining an expected mode of care given a particular set of clinical data;
collecting medical care information relating to the patient using an automated system with access to at least one source of data, including claims data reflecting clinical information relating to the patient obtained from at least one health care provider and submitted in connection with a claim under a health plan;
organizing the collected medical care information into a medical data file for the patient and storing the medical data file, the medical data file comprising patient clinical data, the patient clinical data indicating an actual mode of care provided to the patient;
applying the set of rules to the contents of the medical data file to identify at least one alert based on the patient clinical data, and storing an indicator of the at least one alert in the medical data file;
providing a set of one or more password-protected, personal Web pages for the patient, the set of Web pages including a display of certain elements of the patient's healthcare history automatically populated based on the contents of the medical data file; and
issuing the at least one alert to the patient, via the set of Web pages, automatically-generated based on the presence of the alert indicator in the medical data file, the at least one alert providing an explanation of circumstances underlying the at least one alert and a suggestion for patient follow up.

2. The method of claim 1 wherein the at least one alert is generated when the patient's actual care as indicated by the patient clinical data is inconsistent with an expected mode of care defined by at least one of the rules.

3. The method of claim 1 wherein issuing the at least one alert further comprises providing to the patient, via the set of Web pages, access to relevant health reference information specifically pertaining to the circumstances underlying the at least one alert.

4. The method of claim 1 further comprising:
providing to the patient access to an interactive health risk assessment questionnaire;
receiving a response to the questionnaire from the patient; and
storing information derived from the response in the medical data file.

5. The method of claim 4 wherein access to the questionnaire is provided via the set of Web pages.

6. The method of claim 1 further comprising providing to the patient via the set of Web pages an input section corresponding to the at least one alert, the input section capable of receiving an indication from the patient that the patient has complied with the suggestion for patient follow up.

7. The method of claim 6 further comprising storing information derived from the indication in the medical data file.

8. The method of claim 1 further comprising:
revising the set of rules based on changes in available medical standards, including creating at least one revised rule; and
applying the revised set of rules to the contents of the medical data file to generate the at least one alert by identifying an instance where the patient's actual care as indicated by the patient clinical data is inconsistent with an expected mode of care defined by the revised rule, and storing an indicator of the instance in the medical data file.

9. The method of claim 1 further comprising:
initially establishing a second set of rules, each rule defining available health reference information relating to a particular set of clinical data;
revising the second set of rules based on changes in available health reference information, including creating at least one revised rule defining a new health reference;
applying the revised set of rules to the contents of the medical data file and identifying an instance where the new health reference defined by the revised rule relates to the patient clinical data, and storing an indicator of the instance in the medical data file; and
issuing the at least one alert to the patient, via the set of Web pages, automatically-generated based on the presence of the indicator in the medical data file, the at least one alert providing an explanation of the availability of the new health reference.

10. The method of claim 1 further comprising:
establishing a second set of rules, each rule defining a query relating to a particular set of clinical data;
applying the second set of rules to the contents of the medical data file and, from the second set of rules, identifying at least one relevant query relating to the patient clinical data;
using the identified relevant query to search the contents of a collection of health reference information, the search returning a relevant health reference; and
providing access to the relevant health reference via the set of Web pages.

11. The method of claim 1 wherein the patient clinical data includes at least one health factor capable of varying over time and the set of Web pages includes a display of certain elements of the patient's healthcare history automatically populated based on the contents of the medical data file, including historical information reflecting changes in the health factor over time, the method further comprising:
providing within the set of Web pages a section for input by the patient of additional historical information pertaining to the health factor;
storing the additional historical information in the medical data file; and
providing via the set of Web pages a graphical display showing the trend of the health factor over time.

12. A computer readable medium having stored thereon computer executable instructions for providing a customized alert to an individual patient via a personal health record, the instructions comprising performing the steps of:
accepting an input comprising a set of rules, the rules established from available medical standards, at least one such rule defining an expected mode of care given a particular set of clinical data;
collecting medical care information relating to the patient using an automated system with access to at least one source of data, including claims data reflecting clinical information relating to the patient obtained from at least one health care provider and submitted in connection with a claim under a health plan;
organizing the collected medical care information into a medical data file for the patient and storing the medical data file, the medical data file comprising patient clinical data, the patient clinical data indicating an actual mode of care provided to the patient;
applying the set of rules to the contents of the medical data file and identifying an instance where the patient's actual care as indicated by the patient clinical data is inconsistent with an expected mode of care defined by at least one of the rules, and storing an indicator of the instance in the medical data file;
providing a set of one or more password-protected, personal Web pages for the patient, the set of Web pages including a display of certain elements of the patient's healthcare history automatically populated based on the contents of the medical data file; and
issuing an alert to the patient, via the set of Web pages, automatically-generated based on the presence of the indicator in the medical data file, the alert providing an explanation of circumstances underlying the identified instance and a suggestion for patient follow up.

13. The computer readable medium of claim 12 further comprising instructions for providing to the patient, via the set of Web pages, access to relevant health reference information specifically pertaining to the circumstances underlying the identified instance.

14. The computer readable medium of claim 12 further comprising instructions for:
providing to the patient access, via the set of Web pages, to an interactive health risk assessment questionnaire;
receiving a response to the questionnaire from the patient; and
storing information derived from the response in the medical data file.

15. The computer readable medium of claim 12 further comprising instructions for:
providing to the patient via the set of Web pages an input section corresponding to the alert, the input section capable of receiving an indication from the patient that the patient has complied with the suggestion for patient follow up; and
storing information derived from the indication in the medical data file.

16. A system for providing a customized alert to an individual patient via a personal health record, the system comprising:
a database for maintaining medical care information relating to the patient through access to at least one source of data, including claims data reflecting clinical information relating to the patient obtained from at least one health care provider and submitted in connection with a claim under a health plan;
a care engine for applying a set of rules to the contents of the database and identifying an instance where the patient's actual care as indicated by the patient clinical data is inconsistent with an expected mode of care defined by at least one of the rules, and storing an indicator of the instance in the database, wherein the set of rules is established from available medical standards; and
a Web-based interface for displaying an alert to the patient, the alert automatically-generated based on the presence of the indicator in the database and providing an explanation of circumstances underlying the identified instance and a suggestion for patient follow up.

17. The system of claim 16 wherein the Web-based interface is capable of providing to the patient access to relevant health reference information specifically pertaining to the circumstances underlying the identified instance.

18. The system of claim 16 further comprising an interactive health risk assessment questionnaire for collecting the patient's response to a plurality of predetermined health risk questions, wherein the Web-based interface provides access to the interactive health risk assessment questionnaire.

19. The system of claim 16 wherein the Web-based interface further comprises an alert update interface for receiving input from the patient indicating whether the patient has complied with the suggestion for patient follow up.

20. The system of claim 19 wherein the database is capable of storing the information derived from the indication.
